(19) United States
(12) Patent Application Publication
(10) Pub. No.: US 2025/0005743 A1
(43) Pub. Date: Jan. 2, 2025

(54) METHOD AND SYSTEM FOR IDENTIFYING HORMONE RECEPTOR STATUS

(71) Applicants: MacKay Memorial Hospital, Taipei City (TW); National Central University, Taoyuan City (TW)

(72) Inventors: Jia-Ching WANG, Taoyuan City (TW); Yi-Chiung HSU, Taoyuan City (TW); En-Jhan HUANG, Taoyuan City (TW); Bach-Tung PHAM, Taoyuan City (TW); Phuong-Thi Le, Taoyuan City (TW); Po-Sheng YANG, Taipei City (TW)

(73) Assignees: MacKay Memorial Hospital, Taipei City (TW); National Central University, Taoyuan City (TW)

(21) Appl. No.: 18/216,607

(22) Filed: Jun. 30, 2023

(51) Int. Cl. G06T 7/00; G16H 50/70

(52) U.S. Cl. CPC G06T 7/0012; G16H 50/70; G06T 2207/10056; G06T 2207/30024; G06T 2207/30096

(57) ABSTRACT

Disclosed herein is an improved system and methods implemented by the system for training a model that capable of identifying a hormone receptor status of a subject via the whole slide images (WSIs) of hematoxylin and eosin (H&E) stain of his/her biopsies. The method comprises steps of: (a) obtaining multiple WSIs having known hormone receptor information; (b) dividing each of the WSIs into a plurality of patches; (c) selecting and combining the patches that express the abnormal H&E stain into a combined image; and (d) training a plurality of combined images independently with the aid of the known hormone receptor information of the WSIs, thereby constructing the model. Also disclosed herein is a method for identifying a hormone receptor status of a subject by using the method and model implemented in the present system.

### BACKGROUND OF THE INVENTION

### 1. Field of the Invention

The present disclosure relates to the field of diagnosis and treatment of cancers. More particularly, the disclosed invention relates to methods and systems for determining and identifying a hormone receptor status of a subject based on whole slide images (WSIs) of hematoxylin and eosin (H&E) stains of his/her biopsy, and treating the subject based on the identified hormone receptor status.

### 2. Description of Related Art

Breast cancer (BC) is the most common cancer affecting women worldwide and the most frequent cause of cancer death in women. In 2020, more than 2 million women across the world were diagnosed with BC, and there were more than 0.68 million of deaths from BC globally. Recent advances in early-stage diagnostic approaches, including mammography, magnetic resonance imaging, ultrasound, computerized tomography, positron emission tomography and biopsy, have improved BC-related mortality and morbidity. However, these techniques have limitations such as being expensive and time consuming, therefore cannot be versatilely applied. Developing an efficient and highly sensitive method for diagnosing early-stage BC is urgently needed.

Various biomarkers have been developed for the detection of BC. A majority of invasive breast cancers are hormone receptor-positive—the tumor cells grow in the presence of estrogen (ER) and/or progesterone (PR). Patients with hormone-receptor positive tumors often clinically benefit from receiving hormonal therapies, which target the ER/PR signaling pathways. In the conventional diagnostic workflow, a biopsy sample collected from a patient is thinly sectioned onto microscope slides for staining followed by a visual diagnosis by a pathologist. Generally, hematoxylin and eosin (H&E) staining is used for primary diagnosis, and the immunohistochemistry (IHC) staining is subsequently used for diagnostic confirmation and subtyping to assay the hormone receptor status (HRS) of the biopsy. Though hormone receptor status is a key tool for prognostic purpose and a predictor of endocrine therapy response, the process of HRS determination via visual inspection of slides has limitations. In addition to disadvantages such as expensive and time-consuming, the test output of IHC is expressed in terms of color, which varies due to sample quality, antibody sources and clones, and technician skill levels. Further, pathologists' decision-making process is inherently subjective and can result in human errors. These factors lead to discordance in ERS determination; an estimated 20% of current IHC-based determinations of ER and PR testing may be inaccurate, placing these patients at risk for suboptimal treatment.

In view of the foregoing, there exists in the related art a need for an improved method and system for determining hormone receptor status of a subject via the whole slide images (WSIs) of hematoxylin and eosin (H&E) stain of his/her biopsy.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, the purpose of the present disclosure is to provide a diagnostic system and method that implemented by the system for identifying a hormone receptor status of a subject, such that the efficiency and accuracy in diagnosis of breast cancers can be highly improved.

In one aspect, the present disclosure is directed to a method for building a model for determining hormone receptor status via whole slide images (WSIs) of hematoxylin and eosin (H&E) stain of a biopsy. The method comprises: (a) obtaining a plurality of WSIs of H&E stain of the biopsy, in which each WSIs comprises a hormone receptor information; (b) dividing each of the WSIs of step (a) into a plurality of patches; (c) classifying the normal and abnormal H&E stain in each of the patches of step (b) by performing tiles extraction; (d) selecting and combining the classified patches of step (c) that exhibit the abnormal H&E stain to produce a combined image of each of the WSIs of H&E stain; and (e) training a plurality of combined images independently produced from step (d) with the aid of the hormone receptor information of step (a) thereby establishing the model. In step (a) of this method, the hormone receptor information comprises a positive or negative expression of a hormone receptor selected from the group consisting of an estrogen receptor (ER), a progesterone receptor (PR), and/or a combination thereof.

According to some embodiments of the present disclosure, in step (e) of the present method, the plurality of combined images is trained by performing a vector-regularized complex matrix factorization (CMF) method. The vector-regularized CMF method mainly comprises following steps: (e-1) obtaining a complex matrix from the complex values of each combined images; (e-2) converting the complex matrix into a complex column vector for each combined images; and (e-3) classifying each combined images into the positive or negative expression of the hormone receptor based on the similarities among the complex column vector obtained in step (e-2).

In some working examples, the afore-mentioned step (e-3) is carried out by performing k-nearest neighbors (k-NN) algorithm.

According to some embodiments of the present disclosure, steps (c), (d), and (e) of the present method can be carried out by deep learning algorithms.

According to some embodiments of the present disclosure, the subject has or is suspected of having a breast cancer.

In another aspect, the present disclosure pertains to a method for determining a hormone receptor status based on a whole slide image (WSI) of hematoxylin and eosin (H&E) stain of a biopsy of a subject. The said method comprises: (a) dividing the WSI of H&E stain into a plurality of patches; (b) selecting and combining the patches that exhibit an abnormal H&E stain to produce a test image by performing tiles extraction; and (c) determining the hormone receptor status by processing the test image produced in step (b) within the model established by the aforementioned method. In this method, the hormone receptor status comprises a positive or negative expression of a hormone receptor selected from the group consisting of an estrogen receptor (ER), a progesterone receptor (PR), and/or a combination thereof.

According to some embodiments of the present disclosure, in step (c) of the present method, the test image is processed by performing a vector-regularized complex matrix factorization (CMF) method, which comprises: (c-1) obtaining a complex matrix from the complex values of the test image; (c-2) converting the complex matrix into a complex column vector for the test image; and (c-3) classifying the test image into the positive or negative expression of the hormone receptor based on the absolute distance between the complex column vector of the test image obtained in step (c-2) and those of the combined images in the model established by the aforementioned method.

In some working examples, the afore-mentioned step (c-3) is carried out by performing k-nearest neighbors (k-NN) algorithm.

In preferred embodiments, the hormone receptor status further comprises an expression intensity of the hormone receptor.

Alternatively or optionally, the vector-regularized CMF method further comprises (c-4) determining the expression intensity of the hormone receptor in the test image based on the ratio between complex column vectors of combined images that are corresponding to the positive and negative expression, respectively, in the model established by the aforementioned method.

According to some embodiments of the present disclosure, steps (b) and (c) of the method are carried out by deep learning algorithms.

According to some embodiments of the present disclosure, the subject has or is suspected of having a breast cancer.

In yet another aspect, the present disclosure is directed to a system, which includes an image collecting unit, a server, and a processor, configured to implement the present method.

More specifically, the image collecting unit is configured to collect one or more candidate whole slide images (WSIs) of hematoxylin and eosin (H&E) stain of a biopsy from a subject. The server is configured to store a model established by the afore-mentioned method, and to receive the one or more candidate WSIs of H&E stain transmitted from the image collecting unit. In addition, the processor is programmed with instructions to execute a method for determining the hormone receptor status of the one or more candidate WSIs of H&E stain transmitted from the server, wherein the method comprises: (a) dividing each of the candidate WSIs of H&E stain into a plurality of patches; (b) selecting and combining the patches respectively exhibiting abnormal H&E stains to produce a test image by performing tiles extraction; and (c) determining the hormone receptor status by processing the test image produced in step (b) with the aid of the model stored in the server, wherein the hormone receptor status comprises a positive or negative expression of a hormone receptor selected from the group consisting of an estrogen receptor (ER), a progesterone receptor (PR), and/or a combination thereof.

In some embodiment of the present disclosure, in step (c) of the present method, the test image is processed by performing a vector-regularized complex matrix factorization (CMF) method, which comprises: (c-1) obtaining a complex matrix from the complex values of the test image; (c-2) converting the complex matrix into a complex column vector for the test image; and (c-3) classifying the test image into the positive or negative expression of the hormone receptor based on the absolute distance between the complex column vector of the test image obtained in step (c-2) and those of the combined images in the model stored in the server.

In some working examples, step (c-3) of the present method is carried out by performing k-nearest neighbors (k-NN) algorithm.

In some embodiment of the present disclosure, the hormone receptor status further comprises an expression intensity of the hormone receptor.

Alternatively or optionally, the vector-regularized CMF method further comprises (c-4) determining the expression intensity of the hormone receptor in the test image based on the ratio between complex column vectors of combined images that are corresponding to the positive and negative expression, respectively, within the model stored in the server.

In some working examples, steps (b) and (c) of the present method can be carried out by deep learning algorithms.

In still another aspect, the present disclosure is directed to a method for determining and treating a breast cancer in a subject in need thereof. The method comprises: (a) obtaining a whole slide image (WSI) of hematoxylin and eosin (H&E) stain from a biopsy of the subject; (b) determining a hormone receptor status of the subject by using the aforementioned method; and (c) administering an anti-cancer treatment to the subject based on the hormone receptor status of step (b), wherein the hormone receptor status comprises a positive or negative expression of a hormone receptor selected from the group consisting of an estrogen receptor (ER), a progesterone receptor (PR), and/or a combination thereof, and an expression intensity thereof; the anti-cancer treatment is selected from the group consisting of a surgery, a radiofrequency ablation, a systemic chemotherapy, a transarterial chemoembolization (TACE), an immunotherapy, a targeted drug therapy, a hormone therapy, and a combination thereof.

According to some embodiments of the present disclosure, the subject is a human.

By virtue of the above configuration, the method and system for determining and identifying a hormone receptor status of a subject can be executed in a rapid manner, thereby improving the efficiency and accuracy in diagnosis of breast cancers.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a flow chart of a method 10 according to one embodiment of the present disclosure;

FIG. 2 is a diagram illustrating a system 20 according to another embodiment of the present disclosure; and

FIG. 3 is a flow chart of a method 30 implemented on the system 20 according to another embodiment of the present disclosure.

In accordance with common practice, the various described features/elements are not drawn to scale but instead are drawn to best illustrate specific features/elements relevant to the present invention. Also, like reference numerals and designations in the various drawings are used to indicate like elements/parts.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definition

For convenience, certain terms employed in the specification, examples and appended claims are collected here. Unless defined otherwise, all technical and scientific terms used herein have the same meaning as commonly understood by one of the ordinary skills in the art to which this invention belongs.

The singular forms “a”, “and”, and “the” are used herein to include plural referents unless the context clearly dictates otherwise.

The term “hormone receptor information” as used herein refers to the expression status of one or more hormone receptors including but not limited to, an estrogen receptor (ER), a progesterone receptor (PR), and a combination thereof. According to the present disclosure, the expression status may be a positive or negative expression, and/or an expression intensity of the hormone receptor.

The term “biopsy” or “biopsy specimen” as interchangeably used herein refers to a tissue sample and/or a cells sample taken from anywhere on or in a subject's body, including normal and/or abnormal skin and organs. Practically, biopsies are often obtained for pathology assessment, in which an adequate specimen of the biopsies are prepared and examined under microscopes. According to the present disclosure, the biopsy therefore contains any specimens derived from tumor or cancer tissues, including breast cancer; preferably, the ER/PR-positive breast cancer and the ER/PR-negative breast cancer.

The term “combined image” as used herein refers to an image that has been reorganized and merged after being divided into multiple patches for feature extraction and then being processed to remove featureless ones. According to the present disclosure, the combined images used for training a machine learning model serve as “reference images”, whereas the combined image that obtained from a subject for identifying his/her hormone receptor status serves as a “test image.”

The term “vector-regularized complex matrix factorization (CMF)” as used herein refers to a matrix factorization method on a complex domain for image representation. The real-valued data are transformed into the complex domain, by which the complex-valued matrix is decomposed into two matrices of bases and coefficients that generally derived from solutions to an unconstraint optimization problem in the complex domain. According to the present disclosure, the vector-regularized CMF is used to simplify the complex matrix and to ultimately extract feature vectors from the real image data that highly discriminative.

The term “complex number(s)” and “complex value(s)” are interchangeably used herein to refer an element of a number system that extends the real numbers with a specific element denoted i, called the imaginary unit and satisfying the equation i²=−1; every complex number or complex value can be expressed in the form of a+bi, where a and b are real numbers.

As used herein, the term “treat,” “treating” and “treatment” are interchangeable, and encompasses partially or completely preventing, ameliorating, mitigating and/or managing a symptom, a secondary disorder or a condition associated with breast cancer.

### 2. Description of the Invention

It has been reported that the morphology of a tumor captured in the hematoxylin and eosin (H&E) stain contains predictive signals for the molecular marker status of the tumor, and that a pattern recognition algorithm can be applied to directly determine the molecular marker status from an H&E-stained image. In the field of pattern recognition, it is important that an image is represented in a manner emphasizing relevant information and that a high-dimensional data space is transformed into a low-dimensional feature subspace. The different ways of image representation produce different recognition results. Appropriate representation thus may explicitly express latent structure of the data, and reduce the redundancy and computational cost. The present invention aims to provide improved methods and systems that address the afore-mentioned issue. Furthermore, the present invention also aims at developing an improved method of complex matrix factorization (CMF) for pattern recognition on H&E-stained images, so as to quantify the expression intensities of hormone receptor status.

### 2.1 Method for Building a Model for Hormone Receptor Status Determination

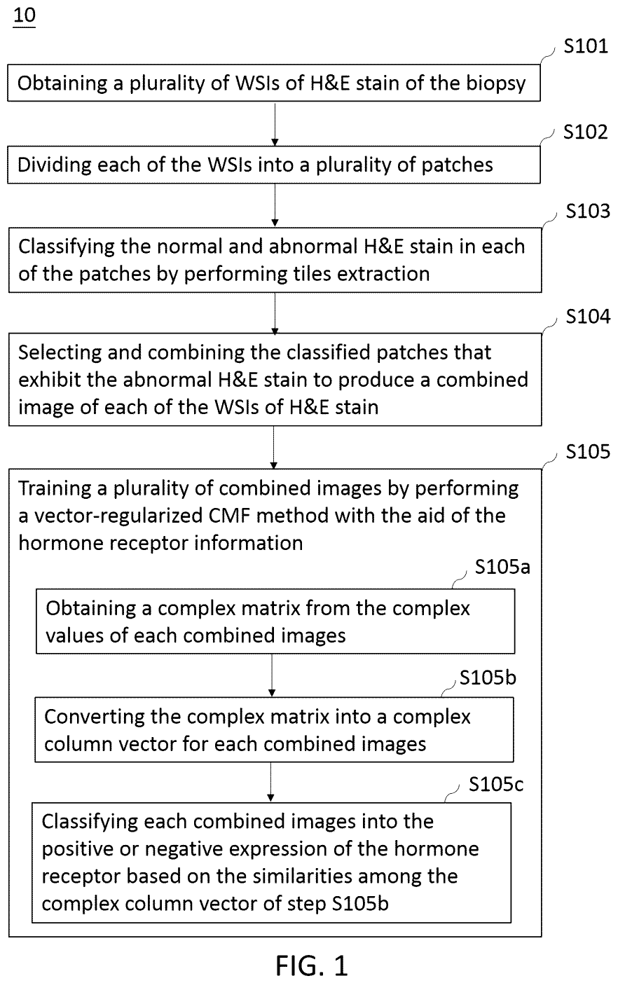

The first aspect of the present disclosure is directed to a method for building a model for determining hormone receptor status via whole slide images (WSIs) of hematoxylin and eosin (H&E) stain of a biopsy. Reference is made to FIG. 1.

FIG. 1 is a flow chart of a method 10 implemented on a computer or a processor according to one embodiment of the present disclosure. The method 10 comprises the following steps, which are respectively indicated by reference numbers S101 to S105 in FIG. 1,
S101: obtaining a plurality of WSIs of H&E stain of the biopsy, in which each WSIs comprises a hormone receptor information;
S102: dividing each of the WSIs of step S101 into a plurality of patches;
S103: classifying the normal and abnormal H&E stain in each of the patches of step S102 by performing tiles extraction;
S104: selecting and combining the classified patches of step S103 that exhibit the abnormal H&E stain to produce a combined image of each of the WSIs of H&E stain; and
S105: training a plurality of combined images independently produced from step S104 with the aid of the hormone receptor information of step S101 thereby establishing the model.

The biopsy of the present method 10 is generally a piece of tissue or a sample of cells obtained from a human body. According to one exemplary embodiment, the biopsy is a piece of breast tissue obtaining from a healthy or a diseased subject. In order to build and train the model, multiple WSIs derived from subjects and independently contain known hormone receptor information are used in the present training method 10. In practice, multiple whole slide images (WSIs) of H&E stain of biopsies may be collected from existing databases of medical centers (S101). According to the present disclosure, the hormone receptor information comprises a positive or negative expression of a hormone receptor selected from the group consisting of an estrogen receptor (ER), a progesterone receptor (PR), and/or a combination thereof. Additionally or optionally, the diagnostic information (e.g., cancer stages) corresponding to each subject may also be collected. Then, the WSIs are automatically forwarded to a device and/or system (e.g., a computer or a processor) having instructions embedded thereon for executing the subsequent steps (S102 to S105). In steps S102 and S103, each of the WSIs is divided into a plurality of patches (i.e., a small piece of the WSI), and each patch is subjected to tile extraction to classify the normal and abnormal H&E stain that exhibits on each of the patches. Tiles extraction can be performed by the algorithms with the aid of preset pathologic criteria well known in the art, so as to distinguish and pick out the patches having abnormal H&E stain from those patches exhibiting normal H&E stains. The classified patches exhibiting the abnormal H&E stain are then subjected to a combination process, which leads to the production of a combined image of each of the WSIs of H&E stain (S104). Note that the patches in one combined image are all derived from one individual, such that each combined image has known hormone receptor information and clinically diagnosed information to refer to when it is subjected to the training process set forth in step S105.

In step S105, a plurality of combined images is then used to train a machine learning algorithm embedded in the computer (e.g., a processor) with the aid of the hormone receptor information mentioned above, thereby establishing the desired model.

According to some embodiments of the present disclosure, the combined images are trained by a vector-regularized complex matrix factorization (CMF) method, which comprises steps indicated by reference numbers S105a to S105c in FIG. 1,
S105a: obtaining a complex matrix from the complex values of each combined images;
S105b: converting the complex matrix into a complex column vector for each combined images; and
S105c: classifying each combined images into the positive or negative expression of the hormone receptor based on the similarities among the complex column vector obtained in step S105b.

Given that each combined image is composed by multiple real numbers per pixels and possesses a real data matrix X of pixels, the ultimate purpose of step S105a is to normalize and transform the real data matrix X into a complex value, thereby yielding a complex matrix Z for one combined image. Note that the complex value in the combined image can be obtained by Fourier transform, and Euler's formula is utilized to convert a point from Cartesian coordinates into polar coordinates. In this scheme, vectors of values of pixel intensity are first normalized and then transformed into the unit sphere using Euler's formula by mapping f from N-dimensional real space to N-dimensional complex space with the aid of equation (1),

$$f ( {x}_{t} ) = {z}_{t} = \frac{1}{\sqrt{2}} {e}^{i απ {x}_{t}} = \frac{1}{\sqrt{2}} [ \begin{matrix}{e}^{i απ {x}_{t} ( 1 )} \\ ⋮ \\ {e}^{i απ {x}_{t} ( N )}\end{matrix} ] , \qquad ( 1 )$$

where xₜ denotes N-dimensional vectors, comprising an expressing image Xₜ in lexicographic ordering, xₜ(c)∈[0,1] and α∈R⁺.

According to the present disclosure, there are N patches (or images) to be trained, and each patch contains M real pixels, which means there are M complex values. By using equation (1), a column vector corresponding to the complex values M of real pixels is obtained (i.e., the right side of the equation (1), as denoted by [ [symbol] ]).

Proceed to step S105b, which aims at obtaining the complex column vector for each combined image from the complex matrix Z. Note that there are N patches and M complex values, the complex matrix Z for a combined image is given by Z∈C^{N×M}. To minimize the objective function, two sub-matrices W∈C^{N×K} and V∈C^{K×M} are factorized from the complex matrix Z, where K denotes a constant. Hence, two sub-matrices W and V are calculated by using the equation (2):

$$f ( W , V ) = \frac{1}{2} {‖ Z - WV ‖}_{F}^{2} + λ {\sum }_{j = 1}^{M} {‖ {V}^{H} {LV}_{: j} ‖}_{1} , \qquad ( 2 )$$

where V^{H}LV represents the complex graph regularization in a real domain (i.e., the combined image), λ is the regularization parameter, Σj₌₁^{M}∥V^{H}LV_{:j}∥₁=Σⱼ₌₁^{M}(Eᵢ₌₁^{K}|Vᵢⱼ|) and a regulates the balance between the accuracy of the factors and the sparseness of matrix V. By disassembling complex matrix Z, both W and V sub-matrices can be learned and obtained after multiple patches (or images) are trained.

Eventually the relationship between the complex matrix Z and the sub-matrices W and V is obtained and denoted by the equations (3) and (4):

$$Z = WV , \qquad ( 3 )$$

$$z = Wv . \qquad ( 4 )$$

Note that the equation (3) can be transformed to equation (4) by substituting z into equation (1), where v represents the complex column vector of the image. Accordingly, the complex column vector v for each combined image is eventually converted from the complex matrix Z or sub-matrix V (S105b). In some preferred embodiments, the complex column vector v is the feature vector of each combined image, thereby allowing further analysis of the combined image via their feature vectors. In sum, by training N patches and disassembling the complex matrix Z, the complex column vector v (i.e., the feature vector) of the combined image is obtained.

Once the complex column vector v is obtained, the vector-regularized CMF method proceeds to the classification step (S105c). In step S105c, every combined image has its complex column vector v and known hormone receptor information including the expression of hormone receptors, thus, by comparing the similarities among multiple complex column vector v, the expression pattern of hormone receptor in the combined image can be classified into either positive or negative groups. Alternatively, the current diagnostic information corresponding to the biopsy source serves the purpose of double confirmation. Hence, with the aid of the known hormone receptor information and/or the diagnostic information in step S101, the images are trained to recognize and classify the positive or negative expression of the hormone receptors, thereby collectively establishing the model for determining hormone receptor status mainly based on H&E stained biopsies. It is worth noting that the algorithm suitable for use in step S105c of the present method can be any well-known classification algorithm. In one working example, step S105c is carried out by performing k-nearest neighbors (k-NN) algorithm.

Training algorithms suitable for use in the present method or system, particularly in steps S103 to S105, may be deep learning algorithms, examples of which may include but are not limited to, convolutional neural networks (CNNs), long short term memory networks (LSTMs), recurrent neural networks (RNNs), generative adversarial networks (GANs), radial basis function networks (RBFNs), multilayer perceptrons (MtLPs), self-organizing maps (SOMs), deep belief networks (DBNs), restricted boltzmann machines (RBMs), and Autoencoders.

By performing the afore-mentioned steps S101 to S105, a model well-trained for determining hormone receptor status directly from H&E stain of biopsies is established.

### 2.2 System and Method for Identifying a Hormone Receptor Status of a Subject

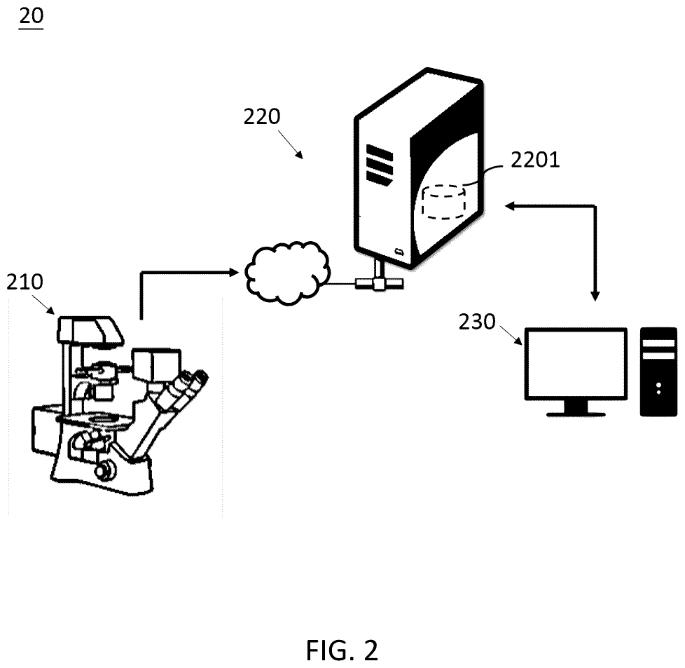

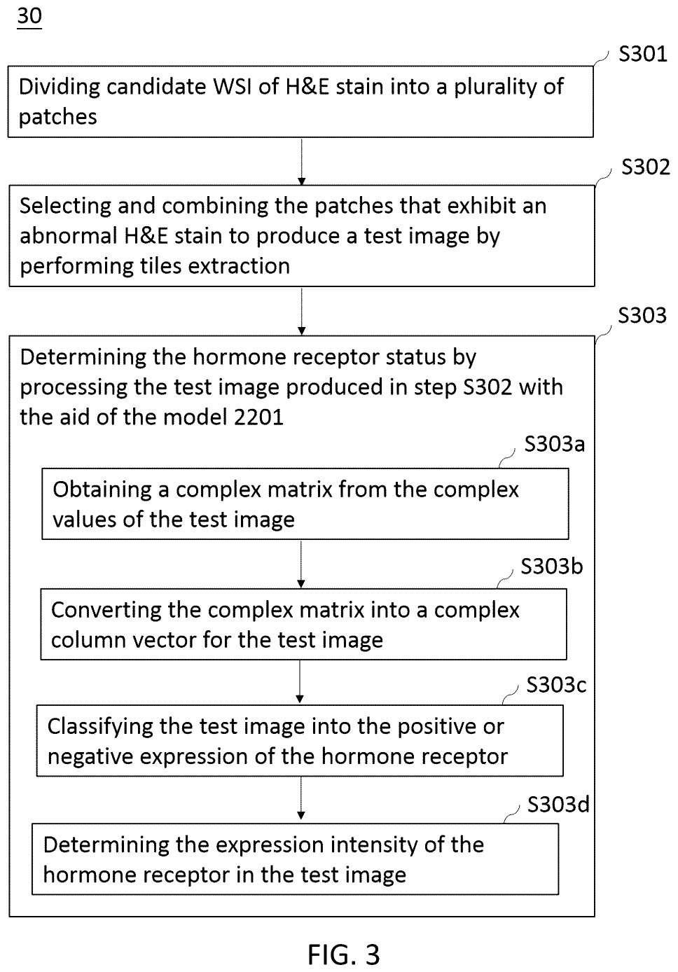

A second aspect of the present disclosure is directed to a method and a system for determining a hormone receptor status based on a candidate WSI of H&E stain of a biopsy collected from a subject. References are made to FIGS. 2 and 3.

FIG. 2 depicts a system 20, which comprises an image collecting unit 210, a server 220, and a processor 230, wherein the image collecting unit 210 and the server 220 are respectively coupled to the processor 230. According to the present disclosure, the image collecting unit 210 is configured to collect one or more candidate WSIs of H&E stain of a biopsy from the subject. In working example, the image collecting unit 210 is a microscopy camera or a whole slide scanner. The server 220 is configured to store a model 2201 established by the present method 10 (i.e., steps S101 to S105) set forth above. The processor 230 is configured to implement image recognition of the present method for hormone receptor status identification.

In some embodiments, the server 220 and the processor 230 are disposed separately as two individual devices; alternatively, they may be disposed in the same hardware. In some embodiments, the server 220 is communicatively connected with the image collecting unit 210 and the processor 230, and configured to store the one or more candidate WSIs of H&E stain that has been received from the image collecting unit 210, and to be analyzed by the processor 230. The processor 230 is programmed with instructions to execute a method for determining the hormone receptor status of the candidate WSIs of H&E stain with the aid of the model 2201 that had built in the server 220.

According to some embodiments of the present disclosure, the image collecting unit 210, the server 220, and the processor 230 are communicatively connected to each other. The communication among the image collecting unit 210, the server 220, and the processor 230 may be embodied using various techniques. For instance, the present server 220 may be a cloud server communicating with the image collecting unit 210 and the processor 230 via a network (such as a local area network (LAN), a wide area network (WAN), the Internet, or a wireless network).

Referring to FIG. 3, which depicts a flow chart of a method 30 implemented on the processor 230 for determining a hormone receptor status of a candidate WSI of H&E stain of a biopsy collected from a subject, who is having or suspected of having a breast cancer. The method 30 includes the following steps (see the reference numbers S301 to S303 indicated in FIG. 3),
S301: dividing the candidate WSI of H&E stain into a plurality of patches;
S302: selecting and combining the patches that exhibit an abnormal H&E stain to produce a test image by performing tiles extraction; and
S303: determining the hormone receptor status by processing the test image produced in step S302 with the aid of the model 2201 established by the present method 10.

According to the present disclosure, the hormone receptor status comprises a positive or negative expression of a hormone receptor. In some alternative embodiments, the hormone receptor status further comprises an expression intensity of the hormone receptor. The hormone receptor for use in the present method is selected from the group consisting of an estrogen receptor (ER), a progesterone receptor (PR), and/or a combination thereof.

Once the candidate WSI of H&E stain is obtained, the processor 230 executes tiles extraction, in which the candidate WSI of H&E stain is divided into a plurality of patches, and then the patches exhibiting an abnormal H&E stain are selected and combined into one test image (steps S301-S302). Like steps S102 and S103 of the method 10, the strategies utilized in steps S301 and S302 can be performed by the algorithms and preset pathologic criteria well known in the art, preferably deep learning algorithms including but are not limited to convolutional neural networks (CNNs), long short term memory networks (LSTMs), recurrent neural networks (RNNs), generative adversarial networks (GANs), radial basis function networks (RBFNs), multilayer perceptrons (MLPs), self organizing maps (SOMs), deep belief networks (DBNs), restricted boltzmann machines (RBMs), and Autoencoders. For the sake of brevity, steps S301 and S302 are not reiterated herein.

Proceed to step S303, the test image is processed and compared with reference information stored in the model 2201, so as to determine the hormone receptor status thereof. According to one embodiment of the present disclosure, the test image is processed by performing a vector-regularized complex matrix factorization (CMF) method conducted by the processor 230. As indicated in FIG. 3, the vector-regularized CMF method generally includes steps of, (S303a) obtaining a complex matrix from the complex values of the test image; (S303b) converting the complex matrix into a complex column vector for the test image; and (S303c) classifying the test image into the positive or negative expression of the hormone receptor based on the absolute distance between the complex column vector of the test image obtained in step S303b and those of the combined images in the model 2201 stored in the server 220.

After obtaining the complex column vector for the test image, classification step (S303c) proceeds. Except step S303c, the strategies utilized in steps S303a and S303b are similar to those described in steps S105a and S105b of the method 10, which also aim at obtaining the feature vector (i.e., a complex column vector) of the real image (i.e., the test image) for image recognition, thereby providing accurate recognition results. Transformation of the real data into complex values is detailed in steps S105a-S105b, therefore is omitted herein for the sake of brevity.

The main difference between step S105c and step S303c is classifying strategy applied to the test image. Unlike step S105, in step S303c, the complex column vector of the test image is exploited to compare with those of the combined images in the model 2201 via calculating the absolute distance therebetween. In general, the closer the distance between complex column vectors of two images, the more similar the images are. Preferably, the said calculation is carried out by performing k-nearest neighbors (k-NN) algorithm. If the complex column vector of the test image is closer to that of the positively expressing combined images than to that of the negatively expressing ones, the complex column vector of the test image is identified as a positively expressing vector. Conversely, if it is closer to the negatively expressing combined images, it is identified as a negatively expressing vector. Practically, in the model 2201, the complex column vector of the test image is compared to those of all the combined images. Each comparison yields a single identification result, producing multiple identification results for the whole comparison of one test image. If the number of complex column vectors corresponding to positive hormone receptor expression is greater than that of negative expression, the test image is determined to possess positive hormone receptor expression. Conversely, if the number of complex column vectors corresponding to negative expression is greater, the test image is determined to exhibit negative hormone receptor expression. Thus, step S303c precisely determines the positive or negative expression of hormone receptors in the test image derived from the subject.

In one preferred embodiment, the vector-regularized CMF method for processing the test image further comprises a step of expression intensity determination (step S303d). Since the comparison in step S303c results in multiple identification numbers for positive and negative hormone receptor expressions, the expression intensity is further calculated as the ratio of these numbers, thereby representing the proportion of positive or negative expressions. For example, if the model has a total of 15 combined images used as reference images, the comparison of these combined images with a test image results in 10 positive and 5 negative expressions out of 15. As a result, the hormone receptor status is considered positive and its expression intensity is represented as 10/15 (10 out of 15 expressions). By doing the calculation, step S303d further determines the intensity of hormone receptor expression in the test images from subjects.

### 2.3 Methods for Determining and Treating Cancers

The present disclosure also aims at providing diagnosis and treatment to a subject afflicted with, or suspected of developing a breast cancer. To this purpose, the afore-described method, model and system may be utilized to assist clinicals with precise determination of hormone receptor status. The present disclosure thus encompasses another aspect that is directed to a method for determining and treating a breast cancer in a subject.

According to some embodiments of the present disclosure, the method comprises,
(a) obtaining a whole slide image (WSI) of hematoxylin and eosin (H&E) stain from a biopsy of the subject;
(b) determining a hormone receptor status of the subject by using the afore-mentioned method and system; and
(c) administering an anti-cancer treatment to the subject based on the hormone receptor status of step (b).

The present method begins by obtaining a whole slide image (WSI) of H&E stain from a biopsy of the subject, which may be a mammal, for example, a human, a mouse, a rat, a hamster, a guinea pig, a rabbit, a dog, a cat, a cow, a goat, a sheep, a monkey, or a horse. Preferably the subject is a human. Suitable tool and/or procedures may be performed to obtain the biopsy and its WSI. In one working example, the biopsy is a breast biopsy stained by hematoxylin and eosin, and its WSI is captured and collected by an image collecting device, such as the image collecting unit 210 (e.g., a microscopy camera or a whole slide scanner) of the present system 20.

Then, the status of hormone receptors of the subject can be determined by the method set forth above. According to the present disclosure, the hormone receptor status comprises a positive or negative expression of a hormone receptor selected from the group consisting of an estrogen receptor (ER), a progesterone receptor (PR), and/or a combination thereof, and an expression intensity thereof.

Once the hormone receptor status of the subject is determined and optionally confirmed, it may then be used as an indicator for the determination of whether an anti-cancer treatment should be administered to the subject. In some embodiments, when the WSI is determined as exhibiting positive ER/PR expression, the subject is likely or having a risk of developing an ER/PR-positive breast cancer, thus anti-cancer treatment is administered to the subject to prevent or ameliorate symptoms associated with the ER/PR-positive breast cancer. In other embodiments, when the WSI is determined as exhibiting negative ER/PR expression, the subject is likely or having a risk of developing an ER/PR-negative breast cancer, thus anti-cancer treatment is administered to the subject to prevent or ameliorate symptoms associated with the ER/PR-negative breast cancer.

Examples of anti-cancer treatments suitable for use in the present method (i.e., for administering to a subject whose hormone receptor status exhibits positive or negative expression) include, but are not limited to, surgery, radiofrequency ablation, systemic chemotherapy, transarterial chemoembolization (TACE), immunotherapy, targeted drug therapy, hormone therapy, and a combination thereof. Any clinical artisans may choose a suitable treatment for use in the present method based on factors such as the particular condition being treated, the severity of the condition, the individual patient parameters (including age, physical condition, size, gender and weight), the duration of the treatment, the nature of concurrent therapy (if any), the specific route of administration and like factors within the knowledge and expertise of the health practitioner.

By the virtue of the above features, the present method can provide precise determination and identification of hormone receptor status mainly based on WSIs of H&E stains without immunohistochemistry (IHC) staining, thereby improving accuracy and efficiency of breast cancer diagnosis and allowing the identified patients to be treated properly.

### EXAMPLES

### Materials and Methods

### Data Collection

A total of 166 estrogen receptor (ER) expressing whole slide images (WSIs) of H&E stain, and a total of 163 progesterone receptor (PR) WSIs of H&E stain of breast biopsies were obtained from the department of breast surgery in Mackay Memorial Hospital (Taipei city) and used for constructing a model of image recognition and verification.

### Image Processing and Tiles Extraction

Every WSIs obtained from the database were rectified into a regularized pixel size of 8× magnification, then divided into 256×256 patches for further deep learning process by utilizing a CNN model.

### Vector-Regularized Complex Matrix Factorization (CMF) for Pattern Recognition

The present vector-regularized CMF method aimed at directly transforming the constrained optimization problem into an unconstrained optimization problem. Based on the principle of Euler's formula, the vectors of values of pixel intensity were normalized and then transformed into the unit sphere by mapping f from N-dimensional real space to N-dimensional complex space with the equation of

$$f ( {x}_{t} ) = {z}_{t} = \frac{1}{\sqrt{2}} {e}^{i απ {x}_{t}} = \frac{1}{\sqrt{2}} [ \begin{matrix}{e}^{i απ {x}_{t} ( 1 )} \\ ⋮ \\ {e}^{i απ {x}_{t} ( N )}\end{matrix} ] , \qquad ( 1 )$$

where xₜ denotes N-dimensional vectors, comprising an expressing image Xₜ in lexicographic ordering, xₜ(c)∈[0, 1] and α∈R⁺.

Next, given a matrix Z∈C^{N×M}, find two matrice W∈C^{N×K} and V∈C^{K×M} that minimized the objective function

$$f ( W , V ) = \frac{1}{2} ‖ Z - WV {‖}_{F}^{2} + λ {\sum }_{j = 1}^{M} {‖ {V}^{H} L {V}_{: j} ‖}_{1} \qquad ( 2 )$$

where λ is the regularization parameter, Σⱼ₌₁^{M}∥V^{H}LV_{:j}∥₁=Σⱼ₌₁^{M}(Σᵢ₌₁^{K}|Vᵢⱼ|), and A regulates the balance between the accuracy of the factors and the sparseness of matrix V.

Notably, ∥Z−WV∥_{F}²=Tr(Z−WV)^{H}(Z−WV)=Tr(Z^{H}Z−2V^{H}W^{H}Z+V^{H}W^{H}WV)

### Example 1 Constructing Image Recognition Model of the Present Disclosure

This experiment aimed at providing a machine learning model trained for WSIs recognition. To this purpose, two models for estrogen receptor (ER) and progesterone receptor (PR) recognition were respectively established in accordance with the procedures set forth in “materials and methods” section. Specifically, for ER recognition (model I), a total of 133 WSIs including 107 WSIs exhibiting a positive ER expression and 26 WSIs exhibiting a negative ER expression were used; and for PR recognition (model II), a total of 130 WSIs including 91 of positive PR expression and 39 of negative PR expression were used.

### Example 2 Evaluation of the Present Image Recognition Model

Next, the image recognition efficiency of the trained model and method for hormone receptor status determination of Example 1 was verified. To this purpose, 33 candidate WSIs including ER and PR expression were processed and input into the present model (i.e., model I and II set forth above) exploiting the present vector-regularized CMF method. In equations (1) and (2), the values of the two parameters α and λ were adjusted in the interval [0,2) and 0.01, respectively.

It was found that the recognition rate for ER identification and PR identification via the present model were 86% and 81%, respectively.

By using the present method and system, the pathological biopsy obtained from patients can be automatically interpreted and identified without additional IHC examination, thereby improving the efficiency and accuracy in breast cancer diagnosis.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for building a model for determining hormone receptor status via whole slide images (WSIs) of hematoxylin and eosin (H&E) stain of a biopsy of a subject, comprising:
(a) obtaining a plurality of WSIs of H&E stain of the biopsy, in which each WSIs comprises a hormone receptor information;
(b) dividing each of the WSIs of step (a) into a plurality of patches;
(c) classifying the normal and abnormal H&E stain in each of the patches of step (b) by performing tiles extraction;
(d) selecting and combining the classified patches of step (c) that exhibit the abnormal H&E stain to produce a combined image of each of the WSIs of H&E stain; and
(e) training a plurality of combined images independently produced from step (d) with the aid of the hormone receptor information of step (a) thereby establishing the model,
wherein the hormone receptor information of step (a) comprises a positive or negative expression of a hormone receptor selected from the group consisting of an estrogen receptor (ER), a progesterone receptor (PR), and/or a combination thereof.

2. The method of claim 1, wherein in step (e), the plurality of combined images is trained by performing a vector-regularized complex matrix factorization (CMF) method, which comprises:
(e-1) obtaining a complex matrix from the complex values of each combined images;
(e-2) converting the complex matrix into a complex column vector for each combined images; and
(e-3) classifying each combined images into the positive or negative expression of the hormone receptor based on the similarities among the complex column vector obtained in step (e-2).

3. The method of claim 2, wherein step (e-3) is carried out by performing k-nearest neighbors (k-NN) algorithm.

4. The method of claim 1, wherein steps (c), (d), and (e) are carried out by deep learning algorithms.

5. The method of claim 1, wherein the subject has or is suspected of having a breast cancer.

6. A method for determining a hormone receptor status based on a whole slide image (WSI) of hematoxylin and eosin (H&E) stain of a biopsy of a subject, comprising:
(a) dividing the WSI of H&E stain into a plurality of patches;
(b) selecting and combining the patches that exhibit an abnormal H&E stain to produce a test image by performing tiles extraction; and
(c) determining the hormone receptor status by processing the test image produced in step (b) within the model established by the method of claim 1,
wherein the hormone receptor status comprises a positive or negative expression of a hormone receptor selected from the group consisting of an estrogen receptor (ER), a progesterone receptor (PR), and/or a combination thereof.

7. The method of claim 6, wherein in step (c), the test image is processed by performing a vector-regularized complex matrix factorization (CMF) method, comprising:
(c-1) obtaining a complex matrix from the complex values of the test image;
(c-2) converting the complex matrix into a complex column vector for the test image; and
(c-3) classifying the test image into the positive or negative expression of the hormone receptor based on the absolute distance between the complex column vector of the test image obtained in step (c-2) and those of the combined images in the model.

8. The method of claim 7, wherein step (c-3) is carried out by performing k-nearest neighbors (k-NN) algorithm.

9. The method of claim 8, wherein the hormone receptor status further comprises an expression intensity of the hormone receptor.

10. The method of claim 9, wherein the vector-regularized CMF method further comprises (c-4) determining the expression intensity of the hormone receptor in the test image based on the ratio between numbers of complex column vectors that are respectively corresponding to the positive and negative expression in the combined images of the model.

11. The method of claim 6, wherein steps (b) and (c) are carried out by deep learning algorithms.

12. The method of claim 6, wherein the subject has or is suspected of having a breast cancer.

13. A system for identifying a hormone receptor status of a subject, comprising:
an image collecting unit configured to collect one or more candidate whole slide images (WSIs) of hematoxylin and eosin (H&E) stain of a biopsy from the subject;
a server configured to store a model established by the method of claim 1, and to receive the one or more candidate WSIs of H&E stain transmitted from the image collecting unit; and
a processor programmed with instructions to execute a method for determining the hormone receptor status of the one or more candidate WSIs of H&E stain transmitted from the server, wherein the method comprises,
(a) dividing each of the candidate WSIs of H&E stain into a plurality of patches;
(b) selecting and combining the patches respectively expressing abnormal H&E stains to produce a test image by performing tiles extraction; and
(c) determining the hormone receptor status by processing the test image produced in step (b) with the aid of the model stored in the server, wherein the hormone receptor status comprises a positive or negative expression of a hormone receptor selected from the group consisting of an estrogen receptor (ER), a progesterone receptor (PR), and/or a combination thereof.

14. The system of claim 13, wherein in step (c) of the method, the test image is processed by performing a vector-regularized complex matrix factorization (CMF) method, comprising:
(c-1) obtaining a complex matrix from the complex values of the test image;
(c-2) converting the complex matrix into a complex column vector for the test image; and
(c-3) classifying the test image into the positive or negative expression of the hormone receptor based on the absolute distance between the complex column vector of the test image obtained in step (c-2) and those of the combined images in the model stored in the server.

15. The system of claim 14, wherein step (c-3) is carried out by performing k-nearest neighbors (k-NN) algorithm.

16. The system of claim 14, wherein the hormone receptor status further comprises an expression intensity of the hormone receptor.

17. The system of claim 16, wherein the vector-regularized CMF method further comprises (c-4) determining the expression intensity of the hormone receptor in the test image based on the ratio between numbers of complex column vectors that are respectively corresponding to the positive and negative expression in the combined images within the model stored in the server.

18. The system of claim 13, wherein steps (b) and (c) are carried out by deep learning algorithms.

19. A method for determining and treating a breast cancer in a subject in need thereof, comprising:
(a) obtaining a whole slide image (WSI) of hematoxylin and eosin (H&E) stain from a biopsy of the subject;
(b) determining a hormone receptor status of the subject by using the method of claim 8; and
(c) administering an anti-cancer treatment to the subject based on the hormone receptor status of step (b),
wherein,
the hormone receptor status comprises a positive or negative expression of a hormone receptor selected from the group consisting of an estrogen receptor (ER), a progesterone receptor (PR), and/or a combination thereof, and an expression intensity thereof; and
the anti-cancer treatment is selected from the group consisting of a surgery, a radiofrequency ablation, a systemic chemotherapy, a transarterial chemoembolization (TACE), an immunotherapy, a targeted drug therapy, a hormone therapy, and a combination thereof.

20. The method of claim 19, wherein the subject is a human.
